(19) United States
(12) Patent Application Publication
(10) Pub. No.: US 2005/0233060 A1
(43) Pub. Date: Oct. 20, 2005

(54) Functional animal muscle protein concentrate composition and process

(76) Inventor: Stephen D. Kelleher, Wakefield, MA (US)

Correspondence Address: Paul J. Cook, 115 Pine Street, Manchester, MA, 01944, US

(21) Appl. No.: 10/655,604

(22) Filed: Sep. 5, 2003

Related U.S. Application Data
(60) Provisional application No. 60/464,614, filed on Apr. 23, 2003.

(51) Int. Cl. C07K 14/47; A23J 1/02; A23J 1/04

(52) U.S. Cl. 426/657; 530/350

(57) ABSTRACT

A meat or fish composition which retains moisture during cooking is provided. A concentrated aqueous acidic protein solution derived from animal muscle tissue is added to the meat or fish prior to cooking. The concentrated aqueous acidic protein solution comprises myofibrillar proteins and sarcoplasmic proteins substantially free of myofibrils and sarcomeres.

### REFERENCE TO RELATED APPLICATION

This application is based on provisional application Ser. No. 60/464,614, filed Apr. 23, 2003.

### BACKGROUND OF THE INVENTION

This invention relates to a process for making an edible functional animal muscle protein concentrate composition; a process for making the composition, a process of using the composition to retain moisture in cooked food and a process for using the composition to preserve food. More particularly, this invention relates to such a protein concentrate composition and process that utilizes animal muscle protein to retain moisture in food, or to preserve food and to the food containing the protein concentrate composition.

Prior to the present invention, protein compositions derived from animal muscle tissue have been available for human consumption as disclosed in U.S. Pat. Nos. 6,005,073; 6,288,216 and 6,451,975 as well as U.S. patent application Ser. No. 10/161,171, filed Jun. 4, 2002. Unfortunately, the liquid form of these compositions are too dilute to permit their addition to foods without compliance to food labeling requirements such as those required by the United States Department of Agriculture (USDA) or the Food and Drug Administration of the United States of America (FDA). Essentially, these labeling requirements, such as “water-added” on the principle display panel, are required when the concentration of water mixed with the active material (protein) is such that the addition of the solution creates a higher ratio of water (or lower ratio of protein) than that which occurs naturally in the food, e.g., fish or meat.

Prior to the present invention, it has been known that meat or fish cooked at an elevated temperature loses its moisture to the surrounding atmosphere. In so doing, the cooked meat or fish undesirably loses its natural or added flavors so that it becomes less tasteful. Fluid loss during cooking of meat or fish can range up to 30% to 40% by weight based upon the weight of the meat or fish prior to cooking. A prior solution for retaining moisture in the meat or fish without additives took the form of wrapping the meat or fish in a solid moisture barrier such as aluminum foil. This solution is undesirable since the surface of the meat or fish remains soft rather than having a desirable crust.

Prior attempts to retain moisture in cooked meat or fish with additives have included the use of sodium tripolyphosphate a coating of fat free flour, based, batter containing an egg white substitute (U.K. Patent Application 2,097,646), water-in-oil emulsion (U.S. Pat. No. 3,406,081), protein or protein isolate and a fat (U.S. Pat. Nos. 4,031,261 and 4,935,251), milk solids (U.S. Pat. No. 2,282,801) and lecithin (U.S. Pat. Nos. 2,470,281 and 3,451,826).

Accordingly, it would be desirable to provide an edible protein composition concentrate derived from animal muscle tissue which can be added directly to a food such as meat or fish without the need for labeling the resultant food product to identify the addition. Furthermore, it would be desirable to provide such a form of fish or meat containing the protein concentrate which is no less nutritional than the original fish or meat to be cooked. In addition, it would be desirable to provide such a form of fish or meat wherein the majority of moisture or added flavors or spices in the uncooked fish or meat is retained during cooking. Furthermore, it would be desirable to provide a form of uncooked meat or fish which is resistant to degradation such as microbial degradation.

### SUMMARY OF THE INVENTION

In accordance with this invention, a concentrated myosin and actin rich composition derived from animal muscle tissue is provided which is in the form of an “aqueous acidic protein solution” as defined below. The animal muscle tissue can contain cholesterol or be free of cholesterol. Also, in accordance with this invention, animal muscle tissue to be cooked is coated or admixed or injected with an aqueous acidic protein solution of a concentrated protein mixture derived from animal muscle tissue comprising a mixture of myofibrillar proteins and sarcoplasmic proteins from a composition obtained by one of the processes disclosed in U.S. Pat. Nos. 6,005,073; 6,288,216; and/or 6,451,975 and/or U.S. patent application Ser. No. 10/161,171, filed Jun. 4, 2002 all of which are incorporated herein by reference in their entirety. Also, in accordance with this invention it has been found that the aqueous solution of the concentrated protein mixture provides a preservative effect against microbial degradation of the food. The compositions of this invention are obtained from the first aqueous acidic solutions described in U.S. Pat. Nos. 6,005,073; 6,288,216; 6,451,975 and application Ser. No. 10/161,171 by subjecting the first aqueous acidic solution to filtration, including microporous filtration (a.k.a. microfiltration), ultrafiltration, reverse osmosis filtration or diafiltration to retain a concentrate protein containing myosin protein and actin protein in the retentate to a protein composition above 0.5%, preferably above 4% by weight protein based on the weight of the solution and recovering the retentate. The retentate solution can be utilized as the aqueous acidic protein solution of this invention

By the phrase “concentrated aqueous acidic protein solution” as used herein is meant a concentrated aqueous solution of myofibrillar proteins and sarcoplasmic proteins derived from animal muscle tissue and having a pH of 3.5 or less and preferably between about 2.5 and about 3.5 but not so low as to adversely affect the protein functionality and which is derived from a dilute acidic aqueous protein solution which is subjected to microporous filtration, ultrafiltration, reverse osmosis filtration or diafiltration under conditions to retain myosin protein and actin protein in the retentate and to pass water into the filtrate. The retentate is recovered as the aqueous acidic protein solution of this invention. The retentate of this invention contains between about 0.5 and about 25 weight percent protein, preferably between about 4 and about 12 weight percent protein based on the weight of the retentate. The aqueous acidic protein solution can be obtained directly from animal muscle tissue by the processes described below followed by filtration with a microporous membrane, an ultrafiltration membrane, a reverse osmosis membrane or a diafiltration membrane.

In accordance with this invention for retaining moisture in cooked meat or fish, the aqueous acidic protein solution of this invention can be injected into the muscle tissue of fish or meat or It can be applied to the surface of the fish or meat or it can be mixed with the fish or meat. The fish or meat containing the concentrated aqueous acidic protein solution then can be cooked at elevated temperature in the absence of a solid moisture barrier while retaining a substantial majority of its original moisture. The difference in weight between meat or fish treated in accordance with this invention compared with fish or meat not injected, mixed or coated with the concentrated aqueous acidic protein solution is between about 4 and about 21%, more usually, between about 4 and about 10%. Also, in accordance with this invention, it has been found that the addition of the concentrated aqueous acidic protein solution of this invention to fish or meat provides a preservative effect in that it reduces microbial degradation of the fish or meat.

### DESCRIPTION OF SPECIFIC EMBODIMENTS

In accordance with this invention, a myosin-rich and actin-rich concentrated protein composition derived from animal muscle is provided in the form of an aqueous acidic protein solution. The animal muscle tissue can contain cholesterol or be free of cholesterol. In addition, in accordance with this invention, animal muscle tissue to be cooked is coated, admixed and/or injected with the concentrated aqueous acidic protein solution. The composition of this invention comprises a mixture of myofibrillar proteins and sarcoplasmic proteins derived from animal muscle tissue and obtained by the processes disclosed in U.S. Pat. Nos. 6,005,073, 6,288,216, Ser. No. 10/161,171, filed Jun. 4, 2002 and U.S. Pat. No. 6,451,975 in the form of a first acidic solution followed filtration with a microporous, ultrafiltration, diafiltration or reverse osmosis membrane of the first aqueous acidic solution to recover the retentate. The retentate is obtained under filtration conditions to recover a protein composition that includes myosin protein and actin protein in the retentate comprising the composition of this invention while directing an aqueous acid and/or salt solution or water into the permeate. In diafiltration, water is added to the protein solution to be filtered in order to carry salts and/or acid through the filter into the filtrate. Water addition is ceased and filtration is continued to reduce the water in the retentate. In reverse osmosis, water, acid and salt are passed through the membrane, thereby increasing protein concentration in the retentate.

The concentrated aqueous acidic protein solution is obtained by one of two processes. In these processes, (acid processes) animal muscle tissue is formed into small tissue particles which are then mixed with sufficient acid to form a solution of the tissue having a pH of 3.5 or less, but not such a low pH as to adversely modify the animal tissue protein, e.g, about 1.0 or less. In one of these two processes, the solution is centrifuged to form a lowest membrane lipid layer, an intermediate layer of aqueous acidic protein solution and a top layer of neutral lipids (fats and oils). The intermediate layer of first aqueous acidic protein solution is then separated from the membrane lipid layer or from both the membrane lipid layer and the neutral lipid layer. In a second of these two processes, the first aqueous acidic protein solution is recovered without a centrifugation step since the starting animal muscle tissue contains low concentrations of undesired lipids, oils and/or fats. In both processes, the protein mixture is free of myofibrils and sarcomeres. In both processes, the first aqueous acidic protein solution is followed by filtration to recover a myosin-rich and actin-rich retentate which comprises the concentrated aqueous acidic protein solution of this invention and to direct an aqueous acid/and or salt solution or water which may or may not contain cholesterol into the permeate. The concentrated aqueous acidic protein solution of this invention contains above 0.5 wt. % protein, preferably to 4 wt. % protein based on the concentrated aqueous acidic protein solution and can be utilized with the uncooked meat or fish. The use of the concentrated aqueous acidic protein solution provides the process advantages of eliminating the prior art steps of raising the pH of the first aqueous acid protein solution followed by a protein precipitation step. The recovered concentrated aqueous acidic protein solution then can be mixed with, injected into or coated on the fish or meat to be cooked or to be preserved.

Filtration can be effected by microporous filtration, ultrafiltration, diafiltration and reverse osmosis filtration. Microporous filtration can be effected with a water wettable microporous membrane such as a membrane designed to retain particles having an average size between about 0.01 and 5 microns. Ultrafiltration can be effected with a water wettable membrane designed to retain particles having an average size between about 0.001 and about 0.02 microns. Reverse osmosis water wettable membranes and diafiltration water wettable membranes are essentially free of pores and, under the process conditions utilized permit passage of water and/or aqueous salt and/or acid solutions through the membrane.

Ultrafiltration is effected with a water wettable ultrafiltration membrane having a molecular weight cut-off which effects retention of myosin heavy chain protein (˜205,000 Daltons) and actin protein (˜42,000 Daltons). Representative suitable ultrafiltration membranes have a molecular weight cut-off between about 3,000 Daltons and about 100,000 Daltons, preferably between about 10,000 Daltons and about 50,000 Daltons. Ultrafiltration membranes having a molecular weight cut-off above 42,000 Daltons can be utilized to retain myosin and actin since the acidic conditions of the solution cause the protein to unfold thereby promoting their retention by the ultrafiltration membranes. Ultrafiltration can be effected by tangential flow filtration (TFF) with a single pass or with multiple passes over the ultrafilter. The retentate recovered during filtration comprises concentrated aqueous acidic protein solution of this invention, which can be utilized directly. The concentrated aqueous acidic protein solution of this invention comprising the retentate has reduced water concentrations, and possibly reduced low molecular weight protein concentrations, as compared to the first aqueous acidic protein solution which is not ultrafiltered. The concentrated aqueous acidic protein solution of this invention contains between about 0.5 and about 25 weight percent protein, preferably between 4 and 12 weight percent, based upon the total weight of the aqueous acidic protein solution. Filtration also can be effected with a reverse osmosis (RO) or diafiltration membrane which permits passage there through of water or an aqueous acid and/or salt solution while retaining proteins. Representative suitable membranes include, polyethersulfones, polyamides, polycarbonates, polyvinylchloride, polyolefins such as polyethylene or polypropylene, cellulose esters such as cellulose acetate or cellulose nitrate, regenerated cellulose, polystyrene, polyimides, polyetherimides, acrylic polymers, methacrylic polymers, copolymers thereof, blends thereof or the like.

Concentrated aqueous acidic protein solution can be applied alone or in admixture with conventional food or nutritive additives such as breading or batter coatings, spice dry rubs, cracker meal, corn meal, spices, flavorings, sugar, salt, pepper or the like. It is preferred to utilize the concentrated aqueous acidic protein solution, with or without food or nutritional additives, for injection. The concentrated aqueous acidic protein solution can be coated on the surface of the meat or fish with an applicator or can be coated by tumbling the meat or fish in the solution or in a marinade containing the acidic aqueous protein solution in a tumbling or vacuum tumbling apparatus.

In summary, the dilute aqueous acidic protein solution utilized in the present invention to form the concentrated aqueous acidic protein solution can be obtained by the following methods:
1. Reduce the pH of comminuted animal muscle tissue to a pH less than about 3.5 to form an acidic protein solution, centrifuge the solution to form a lipid-rich phase and an aqueous phase, recovering a first aqueous acidic protein solution substantially free of membrane lipids and filtering the solution to isolate the retentate comprising the concentrate aqueous acidic protein solution that can be used in this invention.
2. Increase the pH of the aqueous acidic protein solution from method 1 to about pH 5.0-5.5 to effect precipitation of the proteins and then readjust the protein back to a pH of about 4.5 or less using acid in a minimum volume to concentrate the aqueous acidic protein solution to between 3.5-7% protein-and filtering the first acidic protein solution to recover the concentrated aqueous acidic protein solution and retentate.
3. Reduce the pH of comminuted animal muscle tissue to form a first aqueous acidic protein solution which is filtered to produce the concentrated aqueous acidic protein solution of the present invention.

The concentrated aqueous acidic solution is capable of being formed into a gel. The gel is formed by placing the protein into a chopper that is pre-chilled with ice. One part protein (powder) is mixed with 3.7 parts cold water and two (2%) percent NaCl is added to the chopper. The material is adjusted, if necessary, to pH 6.8-7.4. The material is then chopped between 2-3 minutes. The protein prior to cooking should have a moisture content in the 74-82% range. The chopped, protein paste is placed into a polymeric, e.g. polyethylene bag and all the air is removed by hand pressing. The paste is rolled to a thickness of 3 mm and placed for 25 seconds on high in a microwave oven, and then cooled. The final cooled material is tested for its ability to double-fold and rated on a 5-point scale as described by Kudo et al. (1973, Marine Fish. Rev. 32:10-15).

The protein products utilized in the present invention comprise primarily myofibrillar proteins that also contains significant amounts of sarcoplasmic proteins. The sarcoplasmic proteins in the protein product admixed with, injected into or coated on the animal muscle tissue comprises above about 6%, preferably above about 8%, more preferably above about 12 % and most preferably above about 15%, up to about 30% by weight sarcoplasmic proteins, based on the total weight of protein in the dry acidic protein mixture or aqueous acidic protein solution.

In accordance with this invention concentrated aqueous acidic protein solution comprising myofibrillar proteins and sarcoplasmic proteins is applied to the surface of animal muscle to be cooked, or is mixed with the animal muscle tissue to be cooked such as hamburger, sliced reformulated beef or sausage. The term “a surface” as used herein is a surface of the fish or meat which is positioned 90 degrees from an adjacent surface or surfaces of the meat or fish. In addition, the term “a surface” can comprise the connecting surface connecting two adjacent surfaces positioned 90 degrees from each other. Preferably, the entire surface of the meat or fish is coated with the concentrated aqueous acidic protein solution. The coated fish or meat then can be cooked at elevated temperature while retaining a substantial majority of its original moisture.

In one aspect of this invention, particulate meat or fish such as ground meat or fish, e.g. hamburger, is mixed with the concentrated aqueous acidic protein solution comprising myofibrillar proteins and sarcoplasmic proteins at a weight ratio usually comprising about 0.03% to about 15% weight of the protein based on the weight of the uncooked meat or fish, preferably between about 0.5 and 5% weight based on the weight of uncooked meat or fish and most preferably comprising between about 0.5 to about 2% weight based on the weight of the uncooked meat or fish. When the concentrated aqueous acidic protein solution is applied to at least one surface of the meat or fish or it is applied by injection, the amount of the protein mixture added is the same weight ratio as set forth above when mixed with ground meat or fish. When utilizing less than about 0.03% weight concentrated aqueous acidic protein solution, effective moisture retention is not observed. When utilizing greater than about 15% weight dry protein mixture or aqueous acidic protein solution, the cooked meat or fish can become undesirably hard.

It has also been found in accordance with this invention that the addition of the concentrated aqueous acidic protein solution of this invention to uncooked fish or meath provides an unexpected preservative effect in that it reduces degradation by microbes to the food. It is preferred that the concentrated aqueous acidic protein solution be applied to the surface of the food in order to provide this preservation effect.

The animal muscle tissue which is modified in accordance with this invention comprises meat and fish, including shell fish. Representative suitable fish include deboned flounder, sole, haddock, cod, sea bass, salmon, tuna, trout or the like. Representative suitable shell fish include shelled shrimp, crabmeat, crayfish, lobster, scallops, oysters, or shrimp in the shell or the like. Representative suitable meats include ham, beef, lamb, pork, venison, veal, buffalo or the like; poultry such as chicken, mechanically deboned poultry meat, turkey, duck, a game bird or goose or the like either in fillet form or in ground form such as hamburg. The meats can include the bone of the animal when the bone does not adversely affect the edibility of the meat such as spare ribs, lamb chops or pork chops. In addition, processed meat products which include animal muscle tissue such as a sausage composition, a hot dog composition, emulsified product or the like can be coated, injected or mixed with the dry acidic protein mixture, precipitated solid protein mixture and/or the aqueous acidic protein solution, or a combination of these protein addition methods. Sausage and hot dog compositions include ground meat or fish, herbs such as sage, spices, sugar, pepper, salt and fillers such as dairy products as is well known in the art.

The fish or meat containing the concentrated or aqueous acidic protein solution then can be cooked in a conventional manner such as by baking, broiling, deep fat frying, pan frying, in a microwave oven or the like. It has been found that the cooked meat or fish provided in accordance with this invention weighs between about 4% and about 21%, more usually between about 4% and about 9% by weight greater than cooked untreated meat or fish starting from the same uncooked weight.

The following examples illustrate the present invention and are not intended to limit the same.

### EXAMPLE 1

### Ultrafiltration of Myofibrillar & Sarcoplasmic Atlantic Cod Proteins

Fresh Atlantic cod muscle was ground to approximately ⅛th inch and placed into a 5000 ml plastic beaker containing 900 ml cold filtered water (Millipore-Milli DI). The muscle-water mixture was homogenized using a PowerGen 700 homogenizer (Fisher Scientific) on speed 6 for 1 minute. The homogenate was adjusted to pH 2.76 using 2 N hydrochloric acid added drop-wise. The acidified homogenate was centrifuged at 11,000 times gravity force in a Sorvall RC-5B refrigerated centrifuge in a GS-30 rotor for 30 minutes. The protein layer was filtered through four layers of cheesecloth. A 500 ml aliquot was placed into a Millipore Labscale TFF system equipped with a Pellicon XL, PXB050A50, 50,000 Daltons nominal molecular weight cutoff (NWCO), polyethersulfone ultrafiltration membrane. The unit was run in a concentration mode using a feed pressure of 30 psi and a retentate pressure of 10 psi. The starting material was 2.0 Brix % and had a protein concentration of 16.41 mg/ml. After 6 hours and 12 minutes the retentate material was 6.4 Brix % and had a protein concentration of 54.54 mg/ml. The starting material had a moisture content of 97.9% and a cholesterol value of 55.27 mg/100 g. The retentate had a moisture content of 94.3% and no cholesterol was detected. The methods used were AOAC 15^{th} edition 1995.

Approximately 25 ml of the retentate was placed in a small plastic dish and microwaved for 15 seconds and cooled. The resultant material was a soft gel with no residual or loose water. The retentate was also injected using a BD 10 ml syringe (18 gauge needle) into uniformly cut chicken breast pieces. In one chicken piece that weighed 22.26 g, cod protein retentate was injected until a weight of 24.27 g. After microwave cooking the cooked piece had a final weight of 22.78 g. In another piece of chicken the initial weight was 18.45 g and it was injected with cod protein retentate until a weight of 19.70 g. After cooking the final weight was 18.73 g.

### EXAMPLE 2

### Ultrafiltration of Myofibrillar & Sarcoplasmic Pork Proteins

Fresh pork loin muscle was ground to approximately ⅛^{th} inch and placed into a 5000 ml plastic beaker containing 900 ml cold filtered water (Millipore-Milli DI). The muscle-water mixture was homogenized using a PowerGen 700 homogenizer (Fisher Scientific) on speed 6 for 2 minute. The homogenate was adjusted to pH 2.8 using 2 N hydrochloric acid added drop-wise. The acidified homogenate was centrifuged at 11,000 times gravity force in a Sorvall RC-5B refrigerated centrifuge in a GS-30 rotor for 30 minutes. The protein layer was filtered through four layers of cheesecloth. A 500 ml aliquot was placed into a Millipore Labscale TFF system equipped with a Pellicon XL, PXB050A50, 50,000 Daltons NWCO, polyethersulfone ultrafiltration cassette. The unit was run in a concentration mode using a feed pressure of 30 psi and a retentate pressure of 10 psi. The starting material was 1.7 Brix % and had a protein concentration of 18.11 mg/ml. After approximately 12 hours the retentate material was 5.6 Brix % and had a protein concentration of 44.80 mg/ml. The starting material had a moisture content of 98.4% and a cholesterol value of 2.34 mg/100 g. The retentate had a moisture content of 94.9% and no cholesterol was detected. The methods used were AOAC 15^{th} edition 1995. Approximately 20 ml of the retentate was placed in a small plastic dish and microwaved for 30 seconds and cooled. The resultant material was a soft gel with no residual or loose water.

### EXAMPLE 3

### Ultrafiltration of Myofibrillar & Sarcoplasmic Pork Proteins for Use as a Preservative

This example illustrates the formation of the concentrated aqueous acidic protein solution and the use of the solution to perform as a preservative using fresh pork. The concentrated aqueous acidic protein solution was the same material described in Example 2. Fresh, case-ready, pork chops (sell-by-date was the next day) were selected and divided into two groups, treated and controls. The controls were placed into plastic, Ziploc® containers and placed into a refrigerator at 34° F. The treated samples were fully dipped into the above described (5.6 Brix %) retentate and shaken to remove excess protein solution. The treated samples were placed with the controls in refrigerated storage in plastic, Ziploc® containers. All samples were inspected visually, and for odor development every day for a period of 10 days. The controls lasted 2-3 days and the treated samples lasted 9-10 days before the development of off odors.

### EXAMPLE 4

### Diafiltration and Ultrafiltration of Myofibrillar & Sarcoplasmic Pork Proteins

Fresh pork loin muscle (205.28 g) was ground to approximately ⅛^{th} inch a and placed into a 5000 ml plastic beaker and brought to 2000.43 g with cold filtered water (Millipore-Milli DI). The muscle-water mixture was homogenized using a PowerGen 700 homogenizer (Fisher Scientific) on speed 6 for 2 minutes. The homogenate was adjusted to pH 2.64 using 2N hydrochloric acid added drop-wise. The acidified mixture was allowed to set for 20 min. at refrigerated temperatures. The acidified homogenate was centrifuged at10,000 times gravity force in a Sorvall RC-5B refrigerated centrifuge in a GS-30 rotor for 30 minutes. The protein layer was filtered through four layers of cheesecloth. A 500 mi aliquot was placed into a Millipore Labscale TFF system equipped with a Pellicon XL, PXB050A50, 50,000 Daltons NWCO, polyethersulfone ultrafiltration cassette. The unit was run in a diafiltration mode using a feed pressure of 30 psi and a retentate pressure of 10 psi. The diafiltration feed was 5000 ml of chilled cold filtered water (Millipore-Milli D1). After approximately 43 hours the diafiltration mode was stopped consuming all but 72 ml of the original 5000 ml. The unit was then put in concentration mode and concentrated until the retentate material was 5.4 Brix % and had a protein concentration of 31.17 mg/ml. A small portion of the retentate was placed in a small plastic dish and microwaved for 30 seconds and cooled. The resultant material was a soft gel with no residual or loose water.

### EXAMPLE 5

This example illustrates the use of the solution to inject fresh Alaskan Pollock with an injection solution with varying amounts of protein in labeling issues. In order to eliminate the necessity of labeling a food to state that water has been added to the food, it is necessary to add a minimum concentration of protein in the added aqueous solution which is governed by United States Department of Agriculture (USDA), Agricultural Research Services 2002, USDA National Data Base Reference 19 (Nutritional Breakdown).

Injection Solution Scenarios Fresh Alaska Pollock (protein content 16.7%-17.6% mean of 17.2%. Figures in bold denote acceptable scenarios (FDA)

TABLE I

| % Solution | | Final % of |
|---|---|---|
| Injected | % of Protein | Protein in |
| Fish wt. | in Solution | Fillet |
| 4 | 4 | 16.6% |
| 4 | 4.5 | 16.6% |
| 4 | 5 | 16.6% |
| 4 | 5.5 | 16.7% |
| 4 | 6 | 16.8% |
| 4 | 6.5 | 16.8% |
| 4 | 7 | 16.8% |
| 4 | 7.5 | 16.8% |
| 4 | 8 | 16.8% |
| 4 | 8.5 | 16.8% |
| 4 | 9 | 16.9% |
| 4 | 9.5 | 16.9% |
| 4 | 10 | 16.9% |
| 4 | 10.5 | 16.9% |
| 4 | 11 | 16.9% |
| 4 | 11.5 | 17.0% |
| 4 | 12 | 17.0% |

TABLE II

| | | Final % of |
|---|---|---|
| % Solution | % of Protein | Protein in |
| Injected | in Solution | Fillet |
| 4.5 | 4 | 16.6% |
| 4.5 | 4.5 | 16.6% |
| 4.5 | 5 | 16.7% |
| 4.5 | 5.5 | 16.7% |
| 4.5 | 6 | 16.7% |
| 4.5 | 6.5 | 16.7% |
| 4.5 | 7 | 16.7% |
| 4.5 | 7.5 | 16.8% |
| 4.5 | 8 | 16.8% |
| 4.5 | 8.5 | 16.8% |
| 4.5 | 9 | 16.8% |
| 4.5 | 9.5 | 16.8% |
| 4.5 | 10 | 16.9% |
| 4.5 | 10.5 | 16.9% |
| 4.5 | 11 | 16.9% |
| 4.5 | 11.5 | 16.9% |
| 4.5 | 12 | 17.0% |

TABLE III

| | | Final % of |
|---|---|---|
| % Solution | % of Protein | Protein in |
| Injected | in Solution | Fillet |
| 5 | 4 | 16.6% |
| 5 | 4.5 | 16.6% |
| 5 | 5 | 16.6% |
| 5 | 5.5 | 16.6% |
| 5 | 6 | 16.6% |
| 5 | 6.5 | 16.6% |
| 5 | 7 | 16.6% |
| 5 | 7.5 | 16.7% |
| 5 | 8 | 16.7% |
| 5 | 8.5 | 16.8% |
| 5 | 9 | 16.8% |
| 5 | 9.5 | 16.8% |
| 5 | 10 | 16.8% |
| 5 | 10.5 | 16.9% |
| 5 | 11 | 16.9% |
| 5 | 11.5 | 16.9% |
| 5 | 12 | 16.9% |

TABLE IV

| | | Final % of |
|---|---|---|
| % Solution | % of Protein | Protein in |
| Injected | in Solution | Fillet |
| 5.5 | 4 | 16.5% |
| 5.5 | 4.5 | 16.5% |
| 5.5 | 5 | 16.5% |
| 5.5 | 5.5 | 16.6% |
| 5.5 | 6 | 16.6% |
| 5.5 | 6.5 | 16.6% |
| 5.5 | 7 | 16.6% |
| 5.5 | 7.5 | 16.6% |
| 5.5 | 8 | 16.7% |
| 5.5 | 8.5 | 16.7% |
| 5.5 | 9 | 16.8% |
| 5.5 | 9.5 | 16.8% |
| 5.5 | 10 | 16.8% |
| 5.5 | 10.5 | 16.8% |
| 5.5 | 11 | 16.9% |
| 5.5 | 11.5 | 16.9% |
| 5.5 | 12 | 16.9% |

TABLE V

| | | Final % of |
|---|---|---|
| % Solution | % of Protein | Protein in |
| Injected | in Solution | Fillet |
| 6 | 4 | 16.4% |
| 6 | 4.5 | 16.5% |
| 6 | 5 | 16.5% |
| 6 | 5.5 | 16.5% |
| 6 | 6 | 16.5% |
| 6 | 6.5 | 16.6% |
| 6 | 7 | 16.6% |
| 6 | 7.5 | 16.6% |
| 6 | 8 | 16.6% |
| 6 | 8.5 | 16.7% |
| 6 | 9 | 16.7% |
| 6 | 9.5 | 16.7% |
| 6 | 10 | 16.8% |
| 6 | 10.5 | 16.8% |
| 6 | 11 | 16.8% |
| 6 | 11.5 | 16.9% |
| 6 | 12 | 16.9% |

TABLE VI

| | | Final % of |
|---|---|---|
| % Solution | % of Protein | Protein in |
| Injected | in Solution | Fillet |
| 6.5 | 4 | 16.4% |
| 6.5 | 4.5 | 16.4% |
| 6.5 | 5 | 16.4% |
| 6.5 | 5.5 | 16.5% |
| 6.5 | 6 | 16.5% |
| 6.5 | 6.5 | 16.5% |
| 6.5 | 7 | 16.6% |
| 6.5 | 7.5 | 16.6% |
| 6.5 | 8 | 16.6% |
| 6.5 | 8.5 | 16.6% |
| 6.5 | 9 | 16.6% |
| 6.5 | 9.5 | 16.6% |
| 6.5 | 10 | 16.7% |
| 6.5 | 10.5 | 16.8% |
| 6.5 | 11 | 16.8% |
| 6.5 | 11.5 | 16.8% |
| 6.5 | 12 | 16.9% |

TABLE VII

| | | Final % of |
|---|---|---|
| % Solution | % of Protein | Protein in |
| Injected | in Solution | Fillet |
| 7 | 4 | 16.3% |
| 7 | 4.5 | 16.4% |
| 7 | 5 | 16.4% |
| 7 | 5.5 | 16.4% |
| 7 | 6 | 16.4% |
| 7 | 6.5 | 16.5% |
| 7 | 7 | 16.5% |
| 7 | 7.5 | 16.5% |
| 7 | 8 | 16.6% |
| 7 | 8.5 | 16.6% |
| 7 | 9 | 16.6% |
| 7 | 9.5 | 16.6% |
| 7 | 10 | 16.6% |
| 7 | 10.5 | 16.7% |
| 7 | 11 | 16.8% |
| 7 | 11.5 | 16.8% |
| 7 | 12 | 16.8% |

TABLE VIII

| | | Final % of |
|---|---|---|
| % Solution | % of Protein | Protein in |
| Injected | in Solution | Fillet |
| 7.5 | 4 | 16.3% |
| 7.5 | 4.5 | 16.3% |
| 7.5 | 5 | 16.3% |
| 7.5 | 5.5 | 16.4% |
| 7.5 | 6 | 16.4% |
| 7.5 | 6.5 | 16.4% |
| 7.5 | 7 | 16.5% |
| 7.5 | 7.5 | 16.5% |
| 7.5 | 8 | 16.5% |
| 7.5 | 8.5 | 16.6% |
| 7.5 | 9 | 16.6% |
| 7.5 | 9.5 | 16.6% |
| 7.5 | 10 | 16.6% |
| 7.5 | 10.5 | 16.6% |
| 7.5 | 11 | 16.7% |
| 7.5 | 11.5 | 16.8% |
| 7.5 | 12 | 16.8% |

TABLE IX

| | | Final % of |
|---|---|---|
| % Solution | % of Protein | Protein in |
| Injected | in Solution | Fillet |
| 8 | 4 | 16.2% |
| 8 | 4.5 | 16.2% |
| 8 | 5 | 16.3% |
| 8 | 5.5 | 16.3% |
| 8 | 6 | 16.4% |
| 8 | 6.5 | 16.4% |
| 8 | 7 | 16.4% |
| 8 | 7.5 | 16.5% |
| 8 | 8 | 16.5% |
| 8 | 8.5 | 16.5% |
| 8 | 9 | 16.6% |
| 8 | 9.5 | 16.6% |
| 8 | 10 | 16.6% |
| 8 | 10.5 | 16.6% |
| 8 | 11 | 16.7% |
| 8 | 11.5 | 16.8% |
| 8 | 12 | 16.8% |

TABLE X

| | | Final % of |
|---|---|---|
| % Solution | % of Protein | Protein in |
| Injected | in Solution | Fillet |
| 9 | 4 | 16.1% |
| 9 | 4.5 | 16.1% |
| 9 | 5 | 16.2% |
| 9 | 5.5 | 16.2% |
| 9 | 6 | 16.3% |
| 9 | 6.5 | 16.3% |
| 9 | 7 | 16.3% |
| 9 | 7.5 | 16.4% |
| 9 | 8 | 16.4% |
| 9 | 8.5 | 16.5% |
| 9 | 9 | 16.5% |
| 9 | 9.5 | 16.5% |
| 9 | 10 | 16.6% |
| 9 | 10.5 | 16.6% |
| 9 | 11 | 16.6% |
| 9 | 11.5 | 16.6% |
| 9 | 12 | 16.8% |

TABLE XI

| | | Final % of |
|---|---|---|
| % Solution | % of Protein | Protein in |
| Injected | in Solution | Fillet |
| 10 | 4 | 16.0% |
| 10 | 5 | 16.1% |
| 10 | 6 | 16.2% |
| 10 | 7 | 16.3% |
| 10 | 8 | 16.3% |
| 10 | 9 | 16.4% |
| 10 | 10 | 16.5% |
| 10 | 11 | 16.6% |
| 10 | 12 | 16.6% |

TABLE XII

| | | Final % of |
|---|---|---|
| % Solution | % of Protein | Protein in |
| Injected | in Solution | Fillet |
| 11 | 4 | 15.9% |
| 11 | 5 | 16.0% |
| 11 | 6 | 16.1% |
| 11 | 7 | 16.2% |
| 11 | 8 | 16.3% |
| 11 | 9 | 16.4% |
| 11 | 10 | 16.5% |
| 11 | 11 | 16.6% |
| 11 | 12 | 16.6% |

1. A concentrated aqueous acidic protein solution isolated from animal muscle tissue containing between 0.5 and 25 weight % protein based on the weight of the solution and having a pH of about 3.5 or less, said solution capable of being formed into a gel.

2. The solution of claim 1 substantially free of membrane lipids.

3. The solution of any one of claims 1 or 2 being derived from fish muscle tissue.

4. The solution of claim 3 wherein said fish is a pelagic fish.

5. The solution of any one of claims 1 or 2 being derived from poultry muscle tissue.

6. The solution of any one of claims 1 or 2 being derived from beef muscle tissue.

7. A process for recovering a concentrated aqueous acidic protein solution derived from animal muscle tissue and containing between about 0.5 and 25 weight % protein based on the weight of the solution, said solution capable of being formed into a gel which comprises:
forming a protein rich aqueous liquid solution including said protein rich composition and having a pH less than about 3.5 from a particulate form of said animal muscle tissue and an aqueous liquid composition having a pH less than about 3.5 which does not substantially degrade protein of said protein rich composition, centrifuging said protein rich aqueous liquid solution to form a protein rich aqueous phase and a nonaqueous phase containing membrane lipids,
recovering said protein rich aqueous phase,
filtering said protein rich aqueous phase to form a retentate containing myosin protein and actin protein and a filtrate containing aqueous acid solution and
recovering said retentate.

8. The process of claim 7 wherein said pH of said concentrated aqueous acidic protein solution is between about 2.5 and about 3.5.

9. The process of any one of claims 7 or 8 wherein said animal muscle tissue is fish muscle tissue.

10. The process of any one of claims 7 or 8 wherein said animal muscle tissue is chicken muscle tissue.

11. The process of claim 9 wherein said fish muscle tissue is pelagic fish muscle tissue.

12. A process for recovering a concentrated aqueous acidic protein solution derived from animal muscle tissue and containing between about 0.5 and 25 weight % protein based on the weight of the solution, said solution capable of being formed into a gel which comprises:
forming a protein rich aqueous liquid solution including said protein and having a pH less than about 3.5 from a particulate form of said animal muscle tissue and an aqueous liquid composition having a pH less than about 3.5 which does not substantially degrade protein of said protein,
recovering said protein rich aqueous liquid solution,
filtering said protein rich aqueous liquid solution to form a retentate containing myosin protein and actin protein and a filtrate containing aqueous acid solution and
recovering said retentate.

13. The process of claim 12 wherein said pH of said concentrated aqueous acidic protein solution is between about 2.5 and about 3.5.

14. The process of any one of claims 12 or 13 wherein said animal muscle tissue is fish muscle tissue.

15. The process of any one of claims 12 or 13 wherein said animal muscle tissue is chicken muscle tissue.

16. The process of claim 15 wherein said fish muscle tissue is pelagic fish muscle tissue.

17. The process for retaining moisture in uncooked animal muscle tissue during cooking of the animal muscle tissue which comprises:
(a) adding to said uncooked animal muscle tissue a concentrated aqueous acidic protein solution and having a pH of about 3.5 or less and containing between 0.5 and 25 weight % protein based on the weight of the solution, said solution capable of being formed into a gel and obtained from animal muscle tissue by dissolving said tissue in an aqueous acid solution at a pH of 3.5 or less to form a first aqueous acidic protein solution and filtering said first aqueous
acidic protein solution to form a retentate comprising said concentrated aqueous acidic protein solution containing myosin and actin by an adding method selected from the group consisting of applying said retentate to at least one surface of said uncooked animal muscle tissue, mixing said retentate with said uncooked animal muscle tissue, injecting said retentate into said uncooked animal muscle tissue and a combination of at least two of said adding methods,
and (b) cooking said uncooked animal muscle tissue and retentate from step (a).

18. The process of claim 17 wherein the retentate is applied to at least one surface of said uncooked animal muscle tissue

19. The process of claim 18 wherein the retentate is applied to all surfaces of said uncooked animal muscle tissue

20. The process of claim 18 wherein the retentate is mixed with said uncooked animal muscle tissue.
into said uncooked animal muscle tissue.

22. (canceled)

23. (canceled)

24. (canceled)

25. (canceled)

26. (canceled)

27. (canceled)

28. (canceled)

29. (canceled)

30. (canceled)

30. (canceled)

31. The process of any one of claims 17 or 20 wherein said retentate is included in a sausage composition.

32. The process of any one of claims 17 or 20 wherein said retentate is included in a hot dog composition.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. The process of claim 17 wherein said pH is between about 2.5 and 3.5.

62. The process of claim 18 wherein said pH is between about 2.5 and 3.5.

63. The process of claim 19 wherein said pH is between about 2.5 and 3.5.

64. The process of claim 20 wherein said pH is between about 2.5 and 3.5.

65. The process of claim 21 wherein said pH is between about 2.5 and 3.5.

66. (canceled)

67. (canceled)

68. (canceled)

69. (canceled)

70. (canceled)

71. (canceled)

72. (canceled)

73. (canceled)

74. (canceled)

75. The process of claim 31 wherein said pH is between about 2.5 and 3.5.

76. The process of claim 32 wherein said pH is between about 2.5 and 3.5.

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)
